(19) United States
(12) Patent Application Publication
(10) Pub. No.: US 2013/0053718 A1
(43) Pub. Date: Feb. 28, 2013

(54) SYSTEM AND METHOD OF NONCONTACT AND REGIONAL RESPIRATORY DETECTION USING A DEPTH CAMERA

(71) Applicants: Yi-Ping Hung, Taipei (TW); Meng-Chieh Yu, Taipei (TW)

(72) Inventors: Yi-Ping Hung, Taipei (TW); Meng-Chieh Yu, Taipei (TW)

(73) Assignee: NATIONAL TAIWAN UNIVERSITY, Taipei (TW)

(21) Appl. No.: 13/594,185

(22) Filed: Aug. 24, 2012

(30) Foreign Application Priority Data
Aug. 26, 2011 (TW) 100130764

(51) Int. Cl. A61B 5/08; A61B 5/11; A61B 5/113

(52) U.S. Cl. 600/534; 600/529

(57) ABSTRACT

A system for non-contact respiratory detection includes a non-contact respiratory detecting device and a processor. The non-contact respiratory detecting device detects respiratory data or a respiratory model of a user. The processor analyzes the respiratory data or the respiratory model to obtain a respiratory analysis result.

### CROSS-REFERENCE TO RELATED APPLICATIONS

This application claims priority to Taiwan Patent Application No. 100130764, filed on Aug. 26, 2011; the entire contents of which is incorporated by reference in its entirety.

### TECHNICAL FIELD

The present disclosure relates to a system and a method of respiratory detection, and more particularly to a system and a method of non-contact respiratory detection using a depth camera, which can be applied to fields such as health care, medicine, rehabilitation, and family fun.

### BACKGROUND

Respiratory gating and modulation are important. In recent lifestyles, nervousness, anxiety and pressure easily cause autonomic nervous system disorders that make respiration shallow and rapid. Shallow and rapid respiration adversely affect oxygen metabolism and also affect organs and systems of the human body. A good respiratory model is able to enhance physical functions as well as maintain health. Learning a suitable respiratory model is often applied in the treatment of mental issues including anxiety, nervousness, depression and the like.

In the field of health care, medical therapy, and rehabilitation, a patient's physical parameters including respiration are monitored. Generally, specific apparatuses are needed for the detection of respiration in medical therapy. These expensive apparatuses and inconvenient processing are difficult to apply to daily life.

Conventional respiratory detection is mainly done with contact-type detectors including respiratory flow detectors, wearable detectors and the like. An electrode is placed in contact with a user's chest to detect physical parameters such as pulse and respiration. For example, TW Pub. No. 201113002 describes a respiratory detection device that is worn as a watch to detect blood oxygen level using an optical sensor. Respiratory smart clothes for specific populations (e.g. respiratory detection for firemen or sportsmen) exist in overseas market, but they are very expensive and inconvenient to wear.

In recent years, non-contact types of respiratory detection techniques are developing. For example, a heat sensor may be applied to monitor a change of a temperature field, and calculation and determination are performed accordingly; ultrasonic technology may be applied for early detection of respiratory disease in birds and mammalians; and radar-based physiological motion sensors may be used to detect physical parameters including respiration. Ultra wideband technology has been discussed in non-contact type of respiratory detection, but such apparatus are expensive and must be aimed aim at the user's chest to perform the detection.

Conventional respiratory detection techniques and relevant apparatuses are limited to medical therapy and health care applications and are very difficult to apply to daily life because the apparatus are very expensive and complicated processing is needed. Further, conventional techniques merely focus on respiratory detection, and do not have feedback to provide improved respiratory solutions for the user. Thus, available techniques are unsuitable for a general user who desires to learn or adjust his respiratory model. Accordingly, a system for respiratory detection and solution with a simple apparatus as well as simple processing is still needed.

### BRIEF SUMMARY

In an embodiment, a system for non-contact respiratory detection includes a non-contact respiratory detecting device and a processor. The non-contact respiratory detecting device detects respiratory data or a respiratory model of a user. The processor analyzes the respiratory data or the respiratory model to obtain a respiratory analysis result.

In another embodiment, a method for non-contact respiratory detection includes detecting, using a non-contact respiratory detecting device, respiratory data or a respiratory model of a user; analyzing the respiratory data or the respiratory model to obtain a respiratory analysis result; and optionally providing the analysis result to the user via a multi-media feedback device.

### BRIEF DESCRIPTION OF THE DRAWINGS

FIG. 1 illustrates an exemplary system for non-contact respiratory detection.

FIG. 2 illustrates an exemplary embodiment of a system of non-contact respiratory detection.

### DETAILED DESCRIPTION

In an embodiment, a non-contact respiratory detecting device detects alteration of a geometric shape or location of a body of a user to obtain respiratory data and/or a respiratory model of the user. The non-contact respiratory detecting device may detect regional respiratory conditions. The region may be the left lung, right lung, thoracic cavity, abdomen, etc. The respiratory data may include at least one of respiratory frequency, respiratory depth, and inhale/exhale ratio. The respiratory model may include thoracic respiration, abdominal respiration, reverse abdominal respiration, and the like.

The non-contact respiratory detection may be achieved using depth photography technology. With respect to depth photography technology, infrared ray depth sensing technology may be applied to determine a distance between a subject and a lens, and a three-dimensional image can be developed accordingly. In one embodiment, the non-contact respiratory detection device is a depth camera.

The systems and methods of the present disclosure further provide a feedback mechanism such as analyzing, responding, recording, evaluating, introducing and the like to assist the user to understand his/her respiratory conditions and learn suitable respiratory techniques. In contrast with prior approaches, the systems and methods of the present disclosure are suitable for general users in daily life.

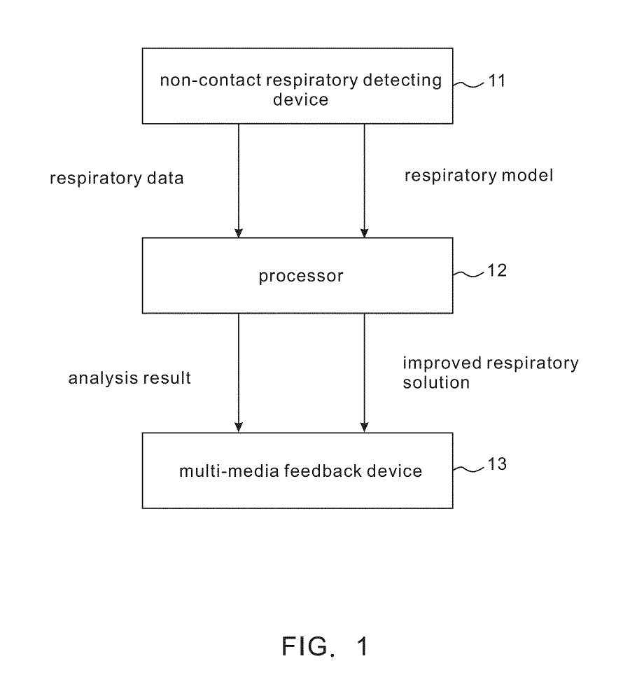

Referring to FIG. 1, an exemplary non-contact respiratory detection system includes a non-contact respiratory detecting device 11, a processor 12 and a multi-media feedback device 13. In the system of FIG. 1, the non-contact respiratory detecting device 11 detects alteration of a geometric shape or location of a body of a user to obtain respiratory data and/or a respiratory model of the user. The respiratory data may include at least one of respiratory frequency, respiratory depth, and inhale/exhale ratio. The respiratory model may include thoracic respiration, abdominal respiration, reverse abdominal respiration, and the like. The respiratory data and/or the respiratory model detected by the non-contact respiratory detecting device 11 is transmitted to the processor 12. The processor 12 analyzes the respiratory data and/or the respiratory model, obtains a respiratory analysis result, selects a feedback mechanism, and determines an improved respiratory solution according to the analysis result. Then, this feedback is provided to the user via the multi-media feedback device 13, such as by showing the analysis result and/or the improved respiratory solution to the user, which is able to assist the user to understand his/her respiratory conditions and learn an improved respiratory solution.

The non-contact respiratory detection device 11 may be a depth sensing device such as, without limitation, an infrared ray depth sensing device. In one preferred embodiment, the non-contact respiratory detection device is a depth camera.

The processor 12 is configured to perform analysis including, without limitation, respiratory model analysis, respiratory condition analysis, abnormal respiratory condition analysis (e.g. sleep apnea), and the like. The processor 12 may include a database to record the analysis result, the respiratory data and the like, so that long-term recording can be achieved and the user can review the records.

The multi-media feedback device 13 may include sensing properties such as image, voice, touch and the like to make the user understand the respiratory information more easily. Further, the multi-media feedback device 13 may assist the user to modulate their respiratory model, improve respiratory habits, and learn a more suitable way of respiration, so that physical functions can be enhanced and improved. In one embodiment, the multi-media feedback device 13 may be selected from a hearing feedback device, a visual feedback device, and a tactile feedback device. In one embodiment, the hearing feedback device may be a stereo set, a speaker, and the like, which can be used for applying voice introduction, sound effects, background sounds/music and the like. In one embodiment, the visual feedback device may be a lighting controller, displays (e.g. displays of computer or cellphone) and the like, which can provide a visual sense to the user such as modulation of environmental light, respiratory pattern, image, text introduction shown by the displays, and the like. In one embodiment, the tactile feedback device may be a heater, a cooler, a vibration motor, a current controller and the like, which generates a feedback to the user via temperature modulation, vibration, miniature current and the like.

In one embodiment, the non-contact respiratory detection system can detect plural users simultaneously, process, analyze and collect information by the processor and the database, so that long-term recording and tracing can be achieved.

It is noted that, in contrast to conventional respiratory detection using an ultra wideband technique, the non-contact respiratory detection system of the present disclosure is able to detect respiratory information of plural users simultaneously with a single device, analyze other activity information as well as body information at the same time, adopt an appropriate algorithm for detection and calculation, and timely respond via a multi-media method/apparatus.

### EXAMPLE

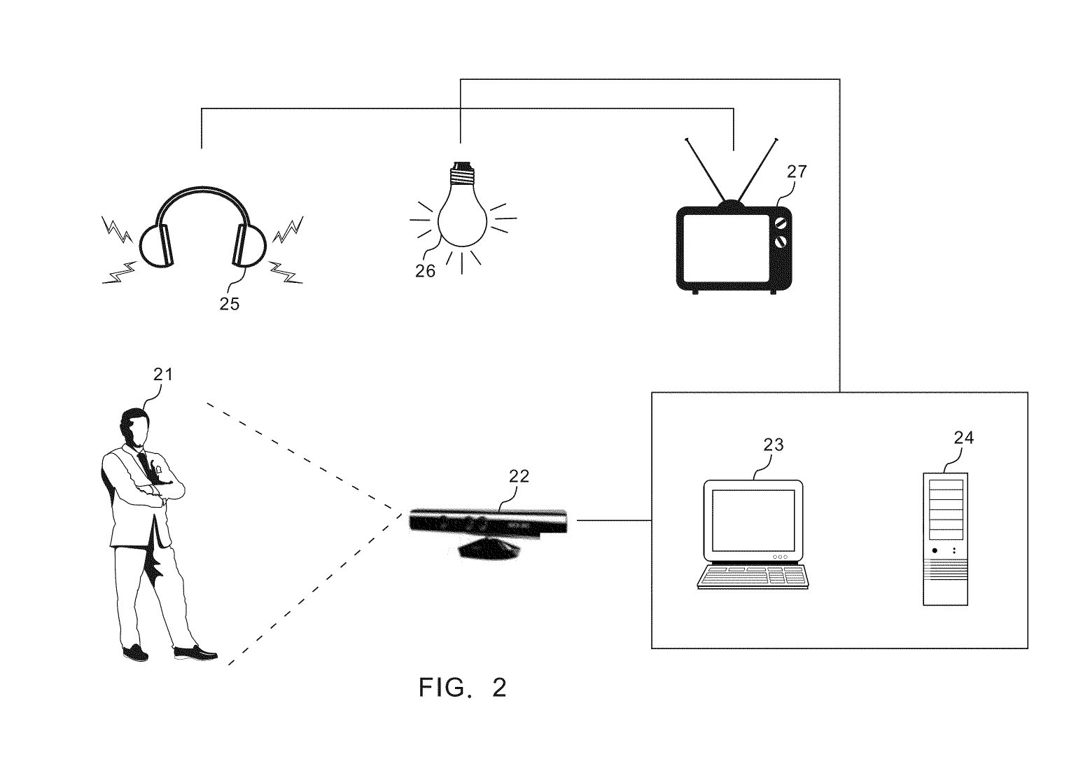

Referring to FIG. 2, a depth camera 22 was used to detect a user 21. In particular, the user's thoracic cavity and abdominal cavity were detected to sense geometric shape alteration, body location alteration and the like. Therefore, respiratory data such as respiratory frequency, respiratory depth, and inhale/exhale ratio, and respiratory model such as thoracic respiration, abdominal respiration, reverse abdominal respiration, fetal respiration and the like were obtained.

In another example, the depth camera 22 was also used to detect a gesture of the user 21. The user 21 put his hand(s) on chest or abdomen, and the body's rising-falling caused by breathing accompanied the location change of his hand(s). The placement of the hand may improve the detection of the gesture changes by the depth camera 22. Other gesture changes of the user 21 caused by breathing may also be detected by the depth camera 22.

Accordingly, gesture detection may be provided as an assistant step, especially for detecting the geometric shape alteration of the user's thoracic cavity and abdominal cavity. Further, analysis of the user's respiratory data and respiratory model may be intensified in response to detecting the gesture.

The detected information was transmitted to the processor 23, and the processor 23 performed signal integration and data analysis to obtain an analysis result such as the user 21 using abdominal respiration, the value of respiratory frequency, the value of inhale/exhale ratio, and the like. Further, a database 24 could provide respiratory information, it also could be used to record the user's long-term respiratory information. All of the above information could be provided to the processor 23 to determine whether an abnormal respiratory condition exists.

The processor 23 further determined and decided an improved respiratory solution according to the analysis result. The improved respiratory solution included, without limitation, increasing depth of breath or reducing respiratory frequency.

Then, based on the selected feedback model, the multi-media feedback information was transmitted to the corresponding feedback device such as the hearing feedback device 25 (such as stereo set, speaker, phonetic system and the like), the lighting controller 26, and the display 27, so that the feedback was provided to the user 21. The feedback included, without limitation, a graph of respiratory information or the improved solution shown by the display 27, or, light modulation (e.g. making the light darker) for relaxing the user and voice introduction for teaching the user to modulate respiratory frequency.

To use the systems and the methods of the present disclosure, the user is not required to wear any respiratory detection apparatus, so the respiratory detection for activity or sleep can be easily performed. Further, while Kinect technology becomes popularized, the price of a depth camera will be significantly reduced to a level that is acceptable to general consumers.

The systems and the methods of the present disclosure have advantages including simple equipment requirements by using a depth camera, a simple and convenient processing step, and real-time detection. In addition, the multi-media feedback mechanism for responding, introducing, evaluating, recording and analyzing in the present system can assist the user to modulate and improve their respiration habits. The present system can be applied to medical therapy and health caring as well as general daily life.

In some embodiments, the described systems and methods may be executed by a special purpose processor/computer or a general purpose processor programmed to execute the process. The described processing and analysis may also be provided in the form of computer executable instructions that, when executed by a processor, cause the processor to execute the processing. The computer executable instructions may be stored on one or more computer readable mediums (e.g., RAM, ROM, etc.) in whole or in parts.

For example, referring to described analysis performed by the processor of FIGS. 1 and 2, some embodiments of a computer or data processing system may include a processor configured to execute at least one program stored in a memory for the purposes of processing data to perform one or more of the techniques that are described herein. The processor may be coupled to a communication interface to receive sensing data. The processor may also receive the sensing data via an input/output block. In addition to storing instructions for the program, the memory may store preliminary, intermediate and final datasets involved in the techniques that are described herein. Among its other features, the computer or data processing system may include a display interface and a display that displays the various data that is generated as described herein. It will be appreciated that the processor shown in FIG. 2 is merely exemplary in nature and is not limiting of the systems and methods described herein.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically and by way of example, a description of a technology in the “Background” is not to be construed as an admission that technology is prior art to any invention(s) in this disclosure. Neither is the “Summary” to be considered as a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall to be considered on their own merits in light of this disclosure, but should not be constrained by the headings set forth herein.

1. A system for non-contact respiratory detection, comprising:
a non-contact respiratory detecting device configured to detect respiratory data or a respiratory model of a user; and
a processor configured to analyze the respiratory data or the respiratory model to obtain a respiratory analysis result.

2. The system of claim 1, wherein the non-contact respiratory detecting device is configured to detect alteration of a geometric shape or location of a body of the user.

3. The system of claim 2, wherein the non-contact respiratory detecting device is a depth camera.

4. The system of claim 2, wherein the alteration of the geometric shape or location of the body includes an alteration of a thoracic cavity, an abdominal cavity, a left lung, a right lung or any combination thereof.

5. The system of claim 1, wherein the respiratory data includes at least one of a respiratory frequency, a respiratory depth, and an inhale/exhale ratio.

6. The system of claim 1, wherein the respiratory model includes at least one of thoracic respiration, abdominal respiration, and reverse abdominal respiration.

7. The system of claim 1, wherein the processor includes a database configured to record the respiratory data and the analysis result.

8. The system of claim 1, wherein the processor is configured to determine an improved solution for the user based on the analysis result, and to select a feedback model for a multi-media feedback device.

9. The system of claim 1, further comprising a multi-media feedback device configured to provide the analysis result to the user.

10. The system of claim 9, wherein the multi-media feedback device includes at least one of a hearing feedback device, a visual feedback device, and a tactile feedback device.

11. The system of claim 10, wherein the visual feedback device includes at least one of a display and a lighting controller.

12. A method for non-contact respiratory detection, comprising:
detecting, using a non-contact respiratory detecting device, respiratory data or a respiratory model of a user;
analyzing the respiratory data or the respiratory model to obtain a respiratory analysis result; and
optionally providing the analysis result to the user via a multi-media feedback device.

13. The method of claim 12, wherein the non-contact respiratory detecting device is configured to detect an alteration of a geometric shape or location of a body of the user.

14. The method of claim 13, wherein the non-contact respiratory detecting device is a depth camera.

15. The method of claim 13, wherein the alteration of the geometric shape or location of the body includes an alteration of a thoracic cavity, an abdominal cavity, a left lung, a right lung or any combination thereof.

16. The method of claim 12, wherein the respiratory data includes at least one of a respiratory frequency, a respiratory depth, and an inhale/exhale ratio.

17. The method of claim 12, wherein the respiratory model includes at least one of thoracic respiration, abdominal respiration, and reverse abdominal respiration.

18. The method of claim 12, further comprising:
detecting, by the non-contact respiratory detecting device, a location alteration of a hand of the user, the location alteration including the user putting the hand on a chest or abdomen of the user; and
intensifying the analysis of the respiratory data or the respiratory model in response to the detecting the location alteration.

19. The method of claim 12, further comprising:
detecting, by a gesture detecting device, a gesture change caused by respiration of the user; and
intensifying the analysis of the respiratory data or the respiratory model in response to the detecting the location alteration.

20. The method of claim 12, wherein the multi-media feedback device includes at least one mechanism configured to respond, introduce, teach, evaluate, record or analyze to assist the user in learning an improved respiratory solution.
